(19) United States
(12) Patent Application Publication
(10) Pub. No.: US 2022/0270722 A1
(43) Pub. Date: Aug. 25, 2022

(54) BEHAVIOR EFFECT ANALYSIS SYSTEM, BEHAVIOR EFFECT ANALYSIS PROGRAM, AND BEHAVIOR EFFECT ANALYSIS METHOD

(71) Applicant: Ryo ISHII, Osaka (JP)

(72) Inventor: Ryo ISHII, Osaka (JP)

(21) Appl. No.: 17/596,731

(22) PCT Filed: Sep. 23, 2020
(86) PCT No.: PCT/JP2020/035839

§ 371 Date: Dec. 16, 2021

(30) Foreign Application Priority Data
Sep. 24, 2019 (JP) 2019-173473

(51) Int. Cl. G16H 10/60

(52) U.S. Cl. CPC G16H 10/60

(57) ABSTRACT

When a behavior history is recorded, an excessive load is imposed on the user because an input operation is required. A technique has been required that simplifies the input operation. A behavior effect analysis system is provided that includes at least: position information acquisition means for acquiring position information; time information acquisition means for acquiring the time at which the user arrives at the position and the staying time at the position; behavior selection means for selecting a behavior executed by the user at the position and storing behavior information; record information acquisition means for acquiring record information including the record value acquired by the user and the record acquisition time; and analysis means for analyzing the effect of each behavior on the record value from the behavior information pertaining to a plurality of behaviors executed within a given period preceding the record acquisition time.

### TECHNICAL FIELD

The present invention relates to a behavior effect analysis system, a behavior effect analysis program and a behavior effect analysis method that analyze an effect of the user's behavior on a record.

### BACKGROUND ART

Systems and apparatuses have been developed that are mainly intended to manage the user's health and record the user's behavior history. Concrete examples include: a data management server in a health management system that links daily life and training together which server presents a recommended activity from the users activity history (see Patent Literature 1); a behavior management system including a list terminal that measures the user's behavior data and a portable terminal that analyzes the behavior data in real time (see Patent Literature 2); a physical condition presentation apparatus that calculates a point based on a specific calculation rule with respect to a behavior executed by the user and presents an evaluation of the physical condition according to the point (see Patent Literature 3); an information processing apparatus that estimates the user's life inactiveness and presents a behavior that the user is highly likely to execute based on the behavior history information (see Patent Literature 4); a monitoring target life monitoring apparatus that compares the detection data of the target of monitoring with the registered life behavior pattern and determines whether the detection data is included in the life behavior pattern data or not (see Patent Literature 5); and a behavior history generation method that generates a behavior history based on behavior contents estimated from action contents identified from a scene extracted from a chronological changing point of the exercise frequency acquired from the user's biological information (Patent Literature 6).

### CITATION LIST

### Patent Literatures

[Patent Literature 1] Japanese Laid-Open Patent Publication No. 2018-45393

[Patent Literature 2] Japanese Laid-Open Patent Publication No. 2017-118550

[Patent Literature 3] Japanese Laid-Open Patent Publication No. 2016-24669

[Patent Literature 4] Japanese Laid-Open Patent Publication No. 2015-49825

[Patent Literature 5] Japanese Laid-Open Patent Publication No. 2004-133777

[Patent Literature 6] WO 2010/032579

### SUMMARY OF INVENTION

### Technical Problem

The conventional systems that record the user's behavior history impose an excessive load on the user because they require an input operation by the user every time a behavior history is recorded. A technique has been required that simplifies the user's input operation and records an accurate behavior history. Moreover, a technique has been required that visualizes the effect of each individual executed behavior on the record based on an accurate behavior history in order for the user to review his/her usual behaviors.

### Solution to Problem

The present invention is a behavior effect analysis system including at least: position information acquisition means for acquiring and storing position information pertaining to a position of a user; time information acquisition means for acquiring and storing a time at which the user arrives at the position and a staying time at the position; behavior selection means for selecting a behavior executed by the user at the position from the position information and the time information, and storing behavior information pertaining to the behavior; record information acquisition means for acquiring and storing record information including a record value pertaining to a record acquired by the user and a record acquisition time; and analysis means for analyzing an effect of each individual behavior on the record value from the behavior information pertaining to a plurality of behaviors executed within a given period preceding the record acquisition time.

Moreover, according to another aspect of the present invention, the behavior information includes at least behavior contents information and the time information associated with the behavior, and the analysis means obtains, by calculation, an effect degree exerted on the record value by the behavior executed during the given period, from the behavior contents information and the time information. The effect degree may be a sum of individual effect degrees exerted on the record value by the individual behaviors. Specifically, the effect degree is expressed as expression 1, and the effect exerted on the record value by each individual behavior is analyzed by comparing the effect degree with the record value.

$${E}_{i , j} = {\sum }_{n = i}^{j} ( {T}_{l a {g}_{n}} \cdot {a}_{n} {f}_{n} ) \qquad [ Expression 1 ]$$

where E_{i,j} is an effect degree from time i to time j,

T_{lag} is a variable obtained from an elapsed time from a behavior executed at n to the record acquisition time,

αₙ is an effect coefficient of the behavior executed at n, and

fₙ is an effect function of the behavior executed at n.

According to another aspect of the present invention, presentation means for presenting a calculation result to the user is further provided, and the presentation means presents, to the user, the effect coefficient obtained by the analysis means performing calculation and/or the effect degree. Moreover, according to still another aspect of the present invention, the analysis means calculates a predicted value of a record that will be acquired by the user next, from the effect degree obtained from the behavior executed during the given period, and the presentation means presents the predicted value to the user.

Moreover, according to another aspect of the present invention, the behavior information includes at least the position information and the time information associated with the behavior, and the behavior selection means compares newly acquired position information and time information with position information and time information stored in the past, selects a behavior associated with close position information and time information from among the position information and the time information stored in the past, and stores behavior information pertaining to the behavior.

Moreover, according to another aspect of the present invention, when there is more than one behavior associated with the close position information and time information, the behavior selection means presents the more than one behavior to the user as choices, and stores behavior information pertaining to the behavior selected by the user.

Further, according to another aspect of the present invention, behavior information of a plurality of users and attribute information of the users are stored and the behavior information includes at least the position information and the time information associated with the behavior, and the behavior selection means compares newly acquired position information and time information with position information and time information associated with a past behavior of an other user having a common attribute with the user, selects a behavior associated with close position information and time information from among the position information and the time information associated with the past behavior of the other user having the common attribute information with the user, and stores behavior information pertaining to the behavior.

Further, the present invention provides a behavior effect analysis program causing a computer to execute. That is, the present invention is a behavior effect analysis program causing a computer to execute: a position information acquisition step of acquiring and storing position information pertaining to a position of a user; a time information acquisition step of acquiring and storing a time at which the user arrives at the position and a staying time at the position; a behavior selection step of selecting a behavior executed by the user at the position from the position information and the time information, and storing behavior information pertaining to the behavior; a record information acquisition step of acquiring and storing record information including a record value pertaining to a record acquired by the user and a record acquisition time; and a step of analyzing an effect of each individual behavior on the record value from the behavior information pertaining to a plurality of behaviors executed within a given period preceding the record acquisition time.

Further, the present invention provides a behavior effect analysis method executed by a computer. That is, the present invention is a behavior effect analysis method including: a position information acquisition step of acquiring and storing position information pertaining to a position of a user; a time information acquisition step of acquiring and storing a time at which the user arrives at the position and a staying time at the position; a behavior selection step of selecting a behavior executed by the user at the position from the position information and the time information, and storing behavior information pertaining to the behavior; a record information acquisition step of acquiring and storing record information including a record value pertaining to a record acquired by the user and a record acquisition time; and a step of analyzing an effect of each individual behavior on the record value from the behavior information pertaining to a plurality of behaviors executed within a given period preceding the record acquisition time.

### Advantageous Effects of Invention

According to the present invention, accurate behavior information can be accumulated by a simple operation by the user. Moreover, since the contribution of each individual behavior on the record value acquired by the user is visualized, an opportunity for the user to review usual behaviors is provided and contribution can be made to improvements in everyday behaviors aiming for improvements in the record.

### BRIEF DESCRIPTION OF DRAWINGS

FIG. 1 is a view showing the structure of an example of a behavior effect analysis system.

FIG. 2 is a view showing the structure of an example of a behavior effect analysis server of the behavior effect analysis system.

### DESCRIPTION OF EMBODIMENTS

A behavior effect analysis system of the present invention is at least provided with position information acquisition means, time information acquisition means, behavior selection means, record information acquisition means, and analysis means. An object of the present invention is to provide an opportunity for the user to review usual behaviors in order to acquire a high record value by presenting the effect of each individual behavior executed by the user on the record value acquired by the user. Hereinafter, each component of the behavior effect analysis system of the present invention will be described in detail.

The position information acquisition means acquires and stores position information pertaining to the position of the user. Specifically, it acquires information transmitted from a position information satellite such as GPS (Global Positioning System). Moreover, it is preferable that more elaborate position information such as a bedroom, a living room, a kitchen, a toilet or a bathroom even in the same building can be acquired by using Near Field Communication such as beacon.

The time information acquisition means acquires and stores the time at which the user arrives at a certain position and the staying time at the position. By the position information acquisition means and the time information acquisition means, when and where the user stayed is stored in storage means provided in the behavior effect analysis system of the present invention.

The behavior selection means selects the behavior executed by the user at the position corresponding to the position information, from the acquired position information and time information, and stores the behavior information pertaining to the behavior.

Here, the behavior information is information indicating what kind of behavior the user executed. The behavior information includes behavior contents information pertaining to the contents of the behavior executed by the user, and the position information and the time information associated with the behavior. That is, at the time point when the behavior is selected, the behavior information pertaining to the behavior is associated with the contents of the behavior, the position where the behavior was executed, the time of arrival at the position and the staying time at the position. The time of arrival at the position and the staying time at the position are estimated to be the time when the selected behavior was executed.

The behavior effect analysis system of the present invention may be provided with a behavior history database. The behavior selection means selects the behavior executed by the user at the position corresponding to the acquired position information, and accumulates the behavior information pertaining to the behavior in the behavior history database. The behavior information accumulated in the behavior history database includes the contents of the user's behavior and the execution position and execution time of the behavior.

Moreover, the behavior selection means is capable of extracting the behavior estimated to have been executed by the user from the position information and the time information with reference to the behavior information accumulated in the previously constructed database, and selecting and storing the extracted behavior information. When there is more than one candidate of the behavior estimated to have been executed by the user as a result of the extraction, the more than one behavior may be presented to the user as choices so that the user selects the correct behavior.

When the executed behavior is uniquely lead from the position information and the time information, the behavior selection means can automatically select and store the behavior information without presenting choices to the user. For example, when the position information indicates a dining room A and the time information indicates that the staying time at the dining room A is from 12 p.m. to 13 p.m., behavior information “lunch (12 to 13, dining room A)” is automatically stored.

The behavior information stored in the database can be changed by the user after stored. For example, when the behavior information “lunch (12 to 13, dining room A)” is stored although the user was working at the dining room A from 12 p.m. to 13 p.m., the user can change the behavior information “lunch (12 to 13, dining room A)” to “work (12 to 13, dining room A)”.

The behavior information includes the position information and the time information associated with the behavior. Further, in another mode of the behavior selection means, the behavior selection means compares the position information and the time information stored in the past with the newly acquired position information and time information, selects the behavior associated with the position information and the time information close to the newly acquired position information and time information, from among the position information and the time information stored in the past, and automatically stores the behavior information pertaining to the behavior. According to the present mode, appropriate behavior information can be automatically stored with no need for the user to select the behavior.

Specifically, based on the acquired position information and time information, the behavior selection means selects the behavior information associated with the position information and the time information close to the acquired position information and time information with reference to the behavior history database. For example, when the position information indicating the dining room A and the time information indicating that the staying time at the dining room A is from 12:05 p.m. to 13:05 p.m. are newly acquired, with reference to the behavior history database, the behavior selection means estimates that the behavior at the dining room A is “work” based on the behavior information “work (12 to 13, dining room A)” stored in the past, stores new behavior information “work (12:05 to 13:05, dining room A)” and accumulates it in the behavior history database.

Further, in a case where there is more than one behavior associated with the close position information and time information when the position information and the time information stored in the past is compared with the newly acquired position information and time information and the behavior associated with the close position information and time information is selected from among the position information and the time information stored in the past, the behavior selection means presents the more than one behavior to the user as choices, and stores the behavior information pertaining to the behavior selected by the user. According to the present mode, the user can select the actually executed behavior from a limited number of choices with a simple operation.

For example, when the position information indicating a dining room B and the time information indicating that the staying time at the dining room B is from 12:15 p.m. to 13:15 p.m. are newly acquired, with reference to the behavior history database, the behavior selection means extracts behavior information “work (12 to 13, dining room B)” and “lunch (11:30 to 12:30, dining room B)” stored in the past. Further, the behavior selection means presents the extracted “work” and “lunch” to the user as choices. When the user selects “lunch”, the behavior selection means accumulates the new behavior information “lunch (12:15 p.m. to 13:15 p.m., dining room B)” in the behavior history database.

The record information acquisition means acquires and stores record information including the record value pertaining to the record acquired by the user and the record acquisition time. The records may be any that can be quantified, and examples thereof include not only athletics records such as the footrace record, the running distance, the number of passes and pass success rate in soccer, and the batting record and pitching record in baseball but also records pertaining to health such as the weight, the body fat rate, the blood pressure, the blood sugar level, the blood cholesterol level, the exercise quantity and calorie consumption amount within a predetermined period and records pertaining to studies and work.

The record information acquisition means accepts the record value inputted from the user and the acquisition time of the record, and accumulates them in the record database. When the record value is the user's biological information (for example, the weight, the body fat ratio or the blood sugar level), the record information acquisition means can automatically acquire the record value and the acquisition time thereof by biological information detection means such as a weight scale, a body fat scale or a glucometer.

The analysis means analyzes the effect of each individual behavior on the record value based on the behavior information pertaining to behaviors executed within a given period preceding the record acquisition time. More specifically, the analysis means obtains the degree of the effect of each individual behavior on the record value by calculation from the effect coefficient preset for each behavior, the behavior contents information and the time information.

Specifically, the analysis means performs the arithmetic processing with the effect coefficients of behaviors contributing to record improvement as positive and the effect coefficients of behaviors contributing to record decline as negative. Moreover, there are cases where even the same behavior is different in the sign of the effect coefficient according to the execution time. For example, having a meal in the morning which contributes to record improvement (for example, improvement in health condition) is positive in the effect coefficient, whereas having a meal during the night which contributes to record decline (for example, decline in health condition) is negative in the effect coefficient.

The user can view a list of the behaviors executed within a given period preceding the record acquisition time. Further, the user can grasp what effect each individual behavior has on the acquired record value. Preferably, the analysis target period is from the record acquisition time immediately preceding the analysis target record acquisition to the analysis target record acquisition time, and the analysis target behaviors are behaviors executed from the record acquisition time immediately preceding the analysis target record acquisition to the analysis target record acquisition time. That is, according to the present invention, it is visualized by which behavior executed during that time, the difference between the analysis target record value and the record value immediately preceding it is effected.

Here, the degree of the effect of each individual behavior on the record value is called individual effect degree. The effect degree obtained by the analysis means is the sum of the individual effect degrees. That is, an effect degree E_{i,j} from time i to time j is obtained as the sum of the individual effect degrees executed from time i to time j and, specifically, expressed as the following expression:

$${E}_{i , j} = {\sum }_{n = i}^{j} ( {T}_{l a {g}_{n}} \cdot {a}_{n} {f}_{n} ) \qquad [ Expression 2 ]$$

When the effect of each individual behavior on the record value is calculated, the individual effect degree may be multiplied by T_{lag} which is a variable obtained from the elapsed time. T_{lag} is set such that behaviors executed in the past near to the record acquisition time have large effects on the record value and behaviors executed in the past distant from the record acquisition time have small effects on the record value. T_{lag} may be individually set according to the kind of the target behavior and the user's characteristics.

The individual effect degree can be calculated by multiplying the effect coefficient by an effect function. The effect coefficient is a coefficient set for each behavior, high values are set as the effect coefficients of behaviors having a large effect on the record value, and low values are set as the effect coefficients of behaviors having a small effect on the record value. The effect function includes one or more than one independent variable. Although not limited, examples of the independent variable referred to here include the number of times of execution of the behavior within a predetermined period time, the time at which the behavior was performed, the heart rate and metal condition during the execution of the behavior and concrete execution contents.

For example, the effect degree E_{i,j} from time i to time j is calculated as the following expression by the variable T_{lag}, an effect coefficient α and an effect function f including independent variables x, y and z:

$${E}_{i , j} = {\sum }_{n = i}^{j} ( {T}_{l a {g}_{n}} \cdot {a}_{n} {f}_{{y}_{n , {z}_{n}}}^{{x}_{n}} ) \qquad [ Expression 3 ]$$

The analysis means of the present invention calculates the above-described effect degree, individual effect degree and effect coefficient. As the calculation means, a known machine learning technique or statistical method may be used. Concrete examples include logistic regression, support vector machine, neural network, multiple regression, support vector regression and partial least squares (PLS) regression.

An example of the calculation of the effect degree, the individual effect degree and the effect coefficient will be shown. A record value Pₜ obtained at time t is, compared with a record value Pₜ₋₁ obtained at (t−1) immediately preceding time t, effected by the behavior executed between t−1 and t. That is, the relationship between the record value P and the effect degree E is shown as the following expression:

Pₜ=E_{t-1,t}+Pₜ₋₁ [Expression 4]

Therefore, the effect degree E can be defined as the following expression as ΔP indicating a change in record value. The effect degree, the individual effect degree and the effect coefficient differ among characteristics of the users. Therefore, by the user each measuring the record value P a plurality of number of times and repeating the calculation by using the machine learning technique or the statistical method according to a plurality of record values P, the effect degree, the individual effect degree and the effect coefficient unique to the user can be obtained.

ΔP=E [Expression 5]

The behavior effect analysis system of the present invention is provided with presentation means for presenting calculation results to the user. The presentation means presents the effect degree, the individual effect degree and the effect coefficient. Further, from the behavior executed during a given period and the individual effect degree and effect coefficient of the behavior, the presentation means calculates the effect degree of the behavior executed during the period, calculates a predicted value of the record that will be acquired by the user next, and presents the predicted value. The presentation means only necessarily presents the calculation results by sound or on a screen, and concrete examples include a display device such as a display and a sound device that provides a presentation by sound.

Moreover, the behavior effect analysis system of the present invention may be provided with a user attribute database storing user attribute information and accumulating the information. By referring to behavior history databases and record databases of a plurality of users having common attribute information, it is possible to perform statistical analysis and set a coefficient such as an effect coefficient common to the users. Moreover, based on the acquired position information and time information, the behavior selection means selects the behavior information associated with close position information and time information with reference to the behavior history databases of a plurality of users including common attribute information. Increase in the information accumulated in the behavior history database enables the behavior selection means to select the users accurate behavior with higher probability.

Further, the present invention provides a behavior effect analysis program that causes a computer to execute the above-described processing. By a computer executing the program, the computer functions as the behavior effect analysis system.

That is, the behavior effect analysis program of the present invention is a behavior effect analysis program that causes a computer to execute: a position information acquisition step of acquiring and storing position information pertaining to the position of the user; a time information acquisition step of acquiring and storing the time at which the user arrives at the position and the staying time at the position; a behavior selection step of selecting the behavior executed by the user at the position from the position information and the time information and storing the behavior information pertaining to the behavior; a record information acquisition step of acquiring and storing record information including the record value pertaining to the record acquired by the user and the record acquisition time; and a step of analyzing the effect of each individual behavior on the record value on the basis of the behavior information pertaining to a plurality of behaviors executed within a given period preceding the record acquisition time.

Further, the present invention provides a behavior effect analysis method by which a computer executes the above-described processing. That is, the behavior effect analysis method of the present invention is a behavior effect analysis method including: a position information acquisition step of acquiring and storing position information pertaining to the position of the user; a time information acquisition step of acquiring and storing the time at which the user arrives at the position and the staying time at the position; a behavior selection step of selecting the behavior executed by the user at the position from the position information and the time information and storing the behavior information pertaining to the behavior; a record information acquisition step of acquiring and storing record information including the record value pertaining to the record acquired by the user and the record acquisition time; and a step of analyzing the effect of each individual behavior on the record value on the basis of the behavior information pertaining to a plurality of behaviors executed within a given period preceding the record acquisition time.

### EXAMPLES

While the present invention will be described in more detail with reference to examples, the present invention is not limited to the examples described below.

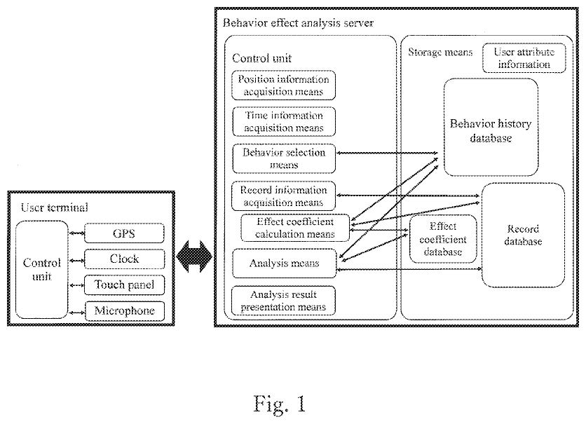

An example of the behavior effect analysis system of the present invention is shown in FIG. 1. The present example includes a user terminal and a behavior effect analysis server capable of being connected to the user terminal through a communication network.

The user terminal preferably has a shape easy to carry for the user, and concrete examples include a portable telephone, a smartphone, a tablet and a wearable device. The user terminal is provided with GPS, a clock, a touch panel and a microphone. The position information and the time information are detected whenever necessary, temporarily stored in a storage unit in the user terminal, and then, transmitted to the behavior effect analysis server. The information inputted by the user through the touch panel or the microphone is also transmitted to the behavior effect analysis server through the communication network.

The behavior effect analysis server is provided with: a control unit having the position information acquisition means, the time information acquisition means, the behavior selection means, the record information acquisition means, the effect coefficient calculation means, the analysis means and the analysis result presentation means; and storage means storing the user attribute information, the behavior history database, the effect coefficient database and the record database.

The position information acquisition means and the time information acquisition means of the behavior effect analysis server acquire and store the position information and the time information transmitted from the user terminal. The behavior selection means selects the behavior estimated to have been executed by the user from the acquired position information and time information with reference to the behavior history database. When the number of selected behaviors is one, the behavior contents, the position and the time are accumulated in the behavior history database in association with one another.

When more than one behavior is selected by the behavior selection means, the behavior effect analysis server displays the more than one behavior as choices on the user terminal through the communication network. The user selects the behavior that he/she actually executed from among the choices by use of the touch panel or the like. The information pertaining to which behavior is selected is transmitted to the behavior effect analysis server through the communication network, and the behavior selection means accumulates the behavior contents, the position and the time in the behavior history database in association with one another.

Further, when the user acquires a record, the user inputs the record value and the record acquisition time of the record by use of the touch panel, the microphone or the like of the user terminal. The inputted record information is transmitted to the behavior effect analysis server through the communication network, and the record information acquisition means accepts the record information and accumulates it in the record database.

The effect coefficient calculation means calculates an effect coefficient with which the effect of the behavior on the record value can be quantified, by statistical analysis referring to the behavior history database and the record database.

The analysis means acquires the record acquisition time of the newly acquired record information and the record acquisition time acquired immediately previously thereto with reference to the record database, and sets the period between the two times as the analysis target period. Further, with reference to the behavior history database, the analysis means reads the information pertaining to the behavior executed during the analysis target period, and with reference to the effect coefficient database, obtains by calculation the probability that each individual executed behaviors provided to the newly accumulated record value, based on the effect coefficient preset for the contents of each executed behavior.

In another example, the analysis means calculates a predicated value of the record that will be acquired by the user next, and presents the predicted value. With reference to the record database, the analysis means acquires the record acquisition time of the latest record information, and sets the period from that time to the time when an inquiry from the user was accepted, as the analysis target period. Further, further, with reference to the behavior history database, the analysis means reads the information pertaining to the behavior executed during the analysis target period, and with reference to the effect coefficient database, obtains by calculation a predicted value of the record that will be acquired by the user next, based on the effect coefficient preset for each individual executed behavior.

The analysis result presentation means transmits the analysis result acquired by the analysis means to the user terminal through a communication network, and displays it on the display of the user terminal.

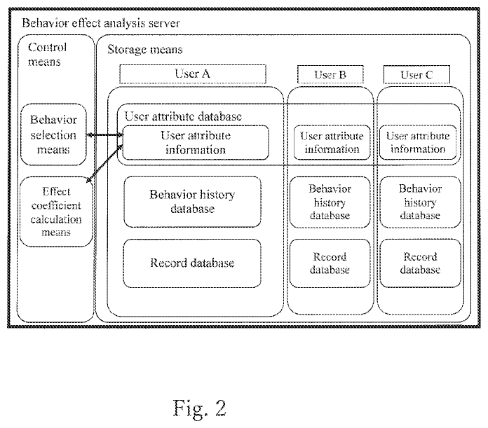

Another example of the behavior effect analysis server constituting the behavior effect analysis system of the present invention is shown in FIG. 2. In the present example, for each of a plurality of users A to C, the user attribute database, the behavior history database and the record database of each user are stored in the storage means of the behavior effect analysis server. When the behavior executed by the user is estimated based on the position information and the time information, the behavior selection means considers the behavior history database of a user having a common attribute with said user with reference to the user attribute database.

Specifically, when selecting the user A's behavior, the behavior selection means searches for a user having a common attribute with the user A with reference to the user attribute database. Suppose the user B and the user A do not have a common attribute and the user C has a common attribute with the user A, the behavior selection means selects the user A's behavior based on the behavior history databases of the user A and the user C.

Likewise, when calculating the effect coefficient, the effect coefficient calculation means of the present example performs statistical analysis including the behavior history database and record database of a user having a common attribute with said user with reference to the user attribute database. Using much information for statistical analysis enables effect coefficient calculation with high accuracy.

In another example, the position information, the time information and the record information are stored in the storage means provided on the user terminal. In the storage means, the user attribute information, the behavior history database, the effect coefficient database and the record database are also stored, so that the above-described processing of behavior selection, effect coefficient calculation and the like can be executed without through the communication network.

1. A behavior effect analysis system comprising at least:
position information acquisition means for acquiring and storing position information pertaining to a position of a user;
time information acquisition means for acquiring and storing a time at which the user arrives at the position and a staying time at the position;
behavior selection means for selecting a behavior executed by the user at the position from the position information and the time information, and storing behavior information pertaining to the behavior;
record information acquisition means for acquiring and storing record information including a record value pertaining to a record acquired by the user and a record acquisition time; and
analysis means for analyzing an effect of each individual behavior on the record value from the behavior information pertaining to a plurality of behaviors executed within a given period preceding the record acquisition time.

2. The behavior effect analysis system according to claim 1,
wherein the behavior information includes at least behavior contents information, and the time information associated with the behavior, and
the analysis means obtains, by calculation, an effect degree exerted on the record value by the behavior executed during the given period, from the behavior contents information and the time information.

3. The behavior effect analysis system according to claim 2,
wherein the effect degree is a sum of individual effect degrees exerted on the record value by the individual behaviors.

4. The behavior effect analysis system according to claim 2,
wherein the effect degree is expressed as
$${E}_{i , j} = {\sum }_{n = i}^{j} ( {T}_{l a {g}_{n}} \cdot {a}_{n} {f}_{n} ) \qquad [ Expression 1 ]$$
where E_{i,j} is an effect degree from time t to time j, T_{lag} is a variable obtained from an elapsed time from a behavior executed at n to the record acquisition time, □ₙ is an effect coefficient of the behavior executed at n, and fₙ is an effect function of the behavior executed at n, and
the effect exerted on the record value by each individual behavior is analyzed by comparing the effect degree with the record value.

5. The behavior effect analysis system according to claim 4, further comprising presentation means for presenting a calculation result to the user,
wherein the presentation means presents, to the user, the effect coefficient obtained by the analysis means performing calculation and/or the effect degree.

6. The behavior effect analysis system according to claim 4, further comprising presentation means for presenting a calculation result to the user,
wherein the analysis means calculates a predicted value of a record that will be acquired by the user next, from the effect degree obtained from the behavior executed during the given period, and
the presentation means presents the predicted value to the user.

7. The behavior effect analysis system according to claim 1,
wherein the behavior information includes at least the position information and the time information associated with the behavior, and
the behavior selection means compares newly acquired position information and time information with position information and time information stored in the past, selects a behavior associated with close position information and time information from among the position information and the time information stored in the past, and stores behavior information pertaining to the behavior.

8. The behavior effect analysis system according to claim 7,
wherein when there is more than one behavior associated with the close position information and time information, the behavior selection means presents the more than one behavior to the user as choices, and stores behavior information pertaining to the behavior selected by the user.

9. The behavior effect analysis system according to claim 1,
wherein behavior information of a plurality of users and attribute information of the users are stored and the behavior information includes at least the position information and the time information associated with the behavior, and
the behavior selection means compares newly acquired position information and time information with position information and time information associated with a past behavior of an other user having a common attribute with the user, selects a behavior associated with close position information and time information from among the position information and the time information associated with the past behavior of the other user having the common attribute information with the user, and stores behavior information pertaining the behavior.

10. A behavior effect analysis program causing a computer to execute:
a position information acquisition step of acquiring and storing position information pertaining to a position of a user;
a time information acquisition step of acquiring and storing a time at which the user arrives at the position and a staying time at the position;
a behavior selection step of selecting a behavior executed by the user at the position from the position information and the time information, and storing behavior information pertaining to the behavior;
a record information acquisition step of acquiring and storing record information including a record value pertaining to a record acquired by the user and a record acquisition time; and
a step of analyzing an effect of each individual behavior on the record value from the behavior information pertaining to a plurality of behaviors executed within a given period preceding the record acquisition time.

11. A behavior effect analysis method executed by a computer, comprising:
a position information acquisition step of acquiring and storing position information pertaining to a position of a user;
a time information acquisition step of acquiring and storing a time at which the user arrives at the position and a staying time at the position;
a behavior selection step of selecting a behavior executed by the user at the position from the position information and the time information, and storing behavior information pertaining to the behavior;
a record information acquisition step of acquiring and storing record information including a record value pertaining to a record acquired by the user and a record acquisition time; and
a step of analyzing an effect of each individual behavior on the record value from the behavior information pertaining to a plurality of behaviors executed within a given period preceding the record acquisition time.
